(19) United States
(12) Patent Application Publication
(10) Pub. No.: US 2019/0192325 A1
(43) Pub. Date: Jun. 27, 2019

(54) Pants with Weight Loss Wrap

(71) Applicant: Leon Flannel, Arlington, TN (US)

(72) Inventor: Leon Flannel, Arlington, TN (US)

(21) Appl. No.: 15/854,853

(22) Filed: Dec. 27, 2017

(51) Int. Cl. A61F 5/00; A41D 15/00; A41C 1/08; A41D 1/089; A41F 9/02

(52) U.S. Cl. CPC A61F 5/0009; A41D 15/002; A41C 1/08; A41D 2300/32; A41F 9/025; A41D 2300/322; A41D 2300/324; A41D 1/089

(57) ABSTRACT

The present disclosure reveals pants with an attached wrap for weight loss and toning one's body. The wrap being comprised of an elastic material that is either impermeable or permeable with pores, wherein the permeable material would allow air to pass through the pores and water, sweat, or other material to pass through or be absorbed into the pores. The wrap may further comprise an absorbent band to absorb sweat.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### DESCRIPTION

### Field of the Invention

It is well known that generally, the population of the United States has an obesity problem. Moreover, many individuals who maintain his or her weight and or include regular workouts into his or her life still have a persistent tummy bulge he or she would like to reduce. This disclosure relates pants, of various length, including but not limited to either shorts, capris length pants, or full-length pants, with an attached weight loss wrap for the purpose of aiding in weight loss in the midriff area of the wearer.

### Background of the Invention

The use of stretchable fabric to aid in weight loss and toning of the body is well known. These disclosures generally include either a belt to be wrapped around the midriff, a full body suit, or a brassier with stretch fabric that extends over the midriff. The present disclosure reveals pants with an attached wrap for the purpose of aiding in shaping a person's appearance and weight loss.

### SUMMARY OF THE INVENTION

The present disclosure reveals pants with an attached wrap for weight loss and toning one's body. The pants comprising but not limited to differing lengths, from shorts, capris length pants, to full length pants. The wrap being comprised of an elastic material that is either impermeable or permeable with pores, wherein the permeable material would allow air to pass through the pores and water, sweat, or other material to pass through or be absorbed into the pores. The impermeable material is comprised of a stretchable fabric, blend of stretchable materials, or layers of stretchable materials common in the industry such as but not limited to sponge neoprene. The permeable material is comprised of a stretchable fabric, blend of stretchable materials, or layers of stretchable materials common in the industry to produce the desired result, such as but not limited to woven polyester.

The wrap is attached to the pants in the area of the upper edge of the pants with the use of an attachment said attachment comprising but not limited to stitching, a touch fastener, snaps, a zipper, or buttons. When the pants are worn, the pants close either close in the usual fashion or are pulled up to the waste of the wearer and held up with the use of a stretchable waste band. The wrap then extends up from the upper edge of the pants and surrounds the midriff of the wearer. The wrap is then closed around the wearer by stretching the wrap so that it encompasses the midriff of the wearer and then attached to itself with the use of a fastener, said fastener comprising but not limited to a touch fastener, snaps, hooks, a zipper, or buttons.

### BRIEF DESCRIPTION OF THE SEVERAL VIEWS OF THE DRAWING

FIG. 1 is a view the pants and wrap wherein the wrap is made from an impermeable material;

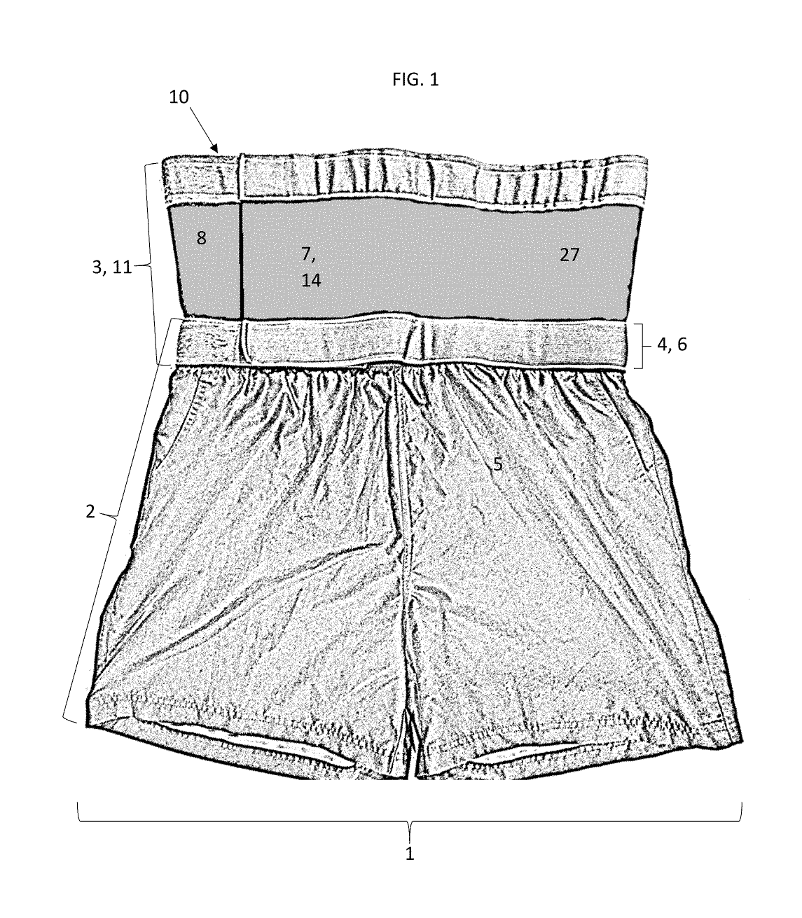

FIG. 2 is a view the pants and wrap wherein the wrap is made from a permeable material;

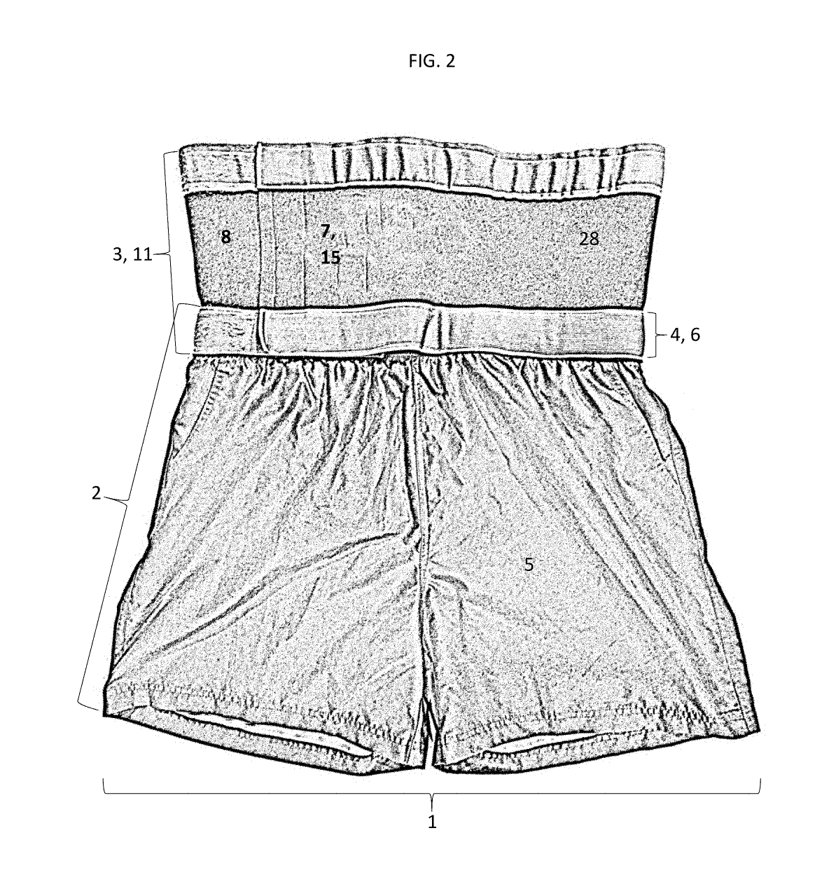

FIG. 3A is a view of the attachment of the wrap to the pants, said attachment being stitching;

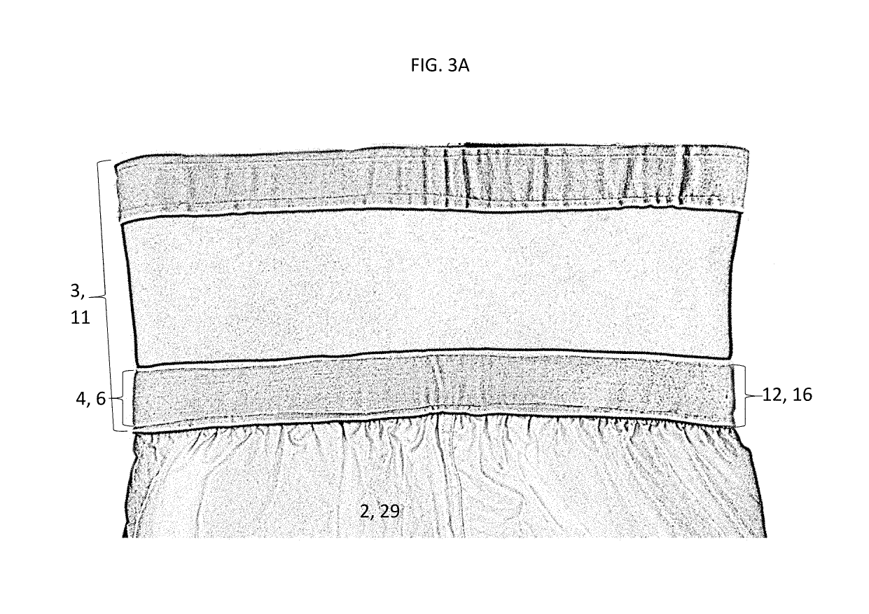

FIG. 3B is a view of the attachment of the wrap to the pants, said attachment being adhesive;

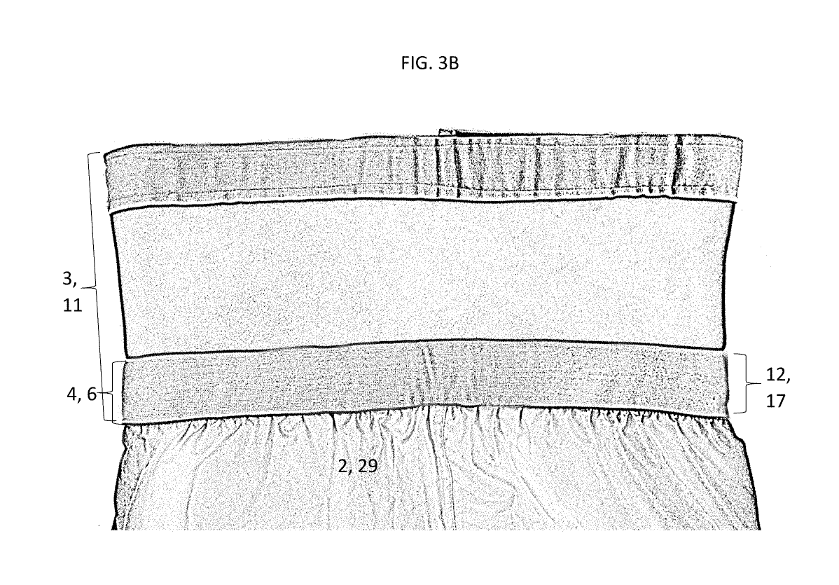

FIG. 3C is a view of the attachment of the wrap to the pants, said attachment being a touch fastener;

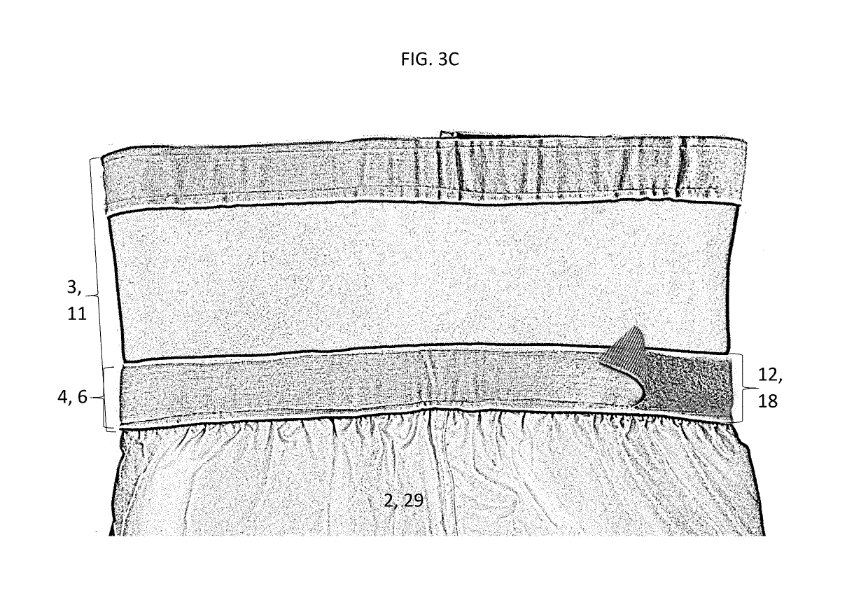

FIG. 3D is a view of the attachment of the wrap to the pants, said attachment being snaps;

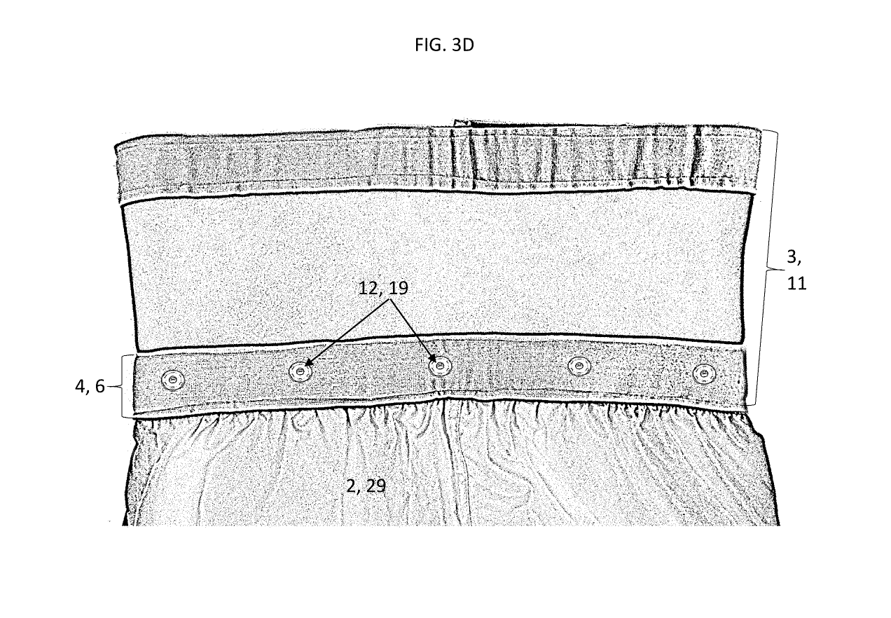

FIG. 3E is a view of the attachment of the wrap to the pants, said attachment being zipper;

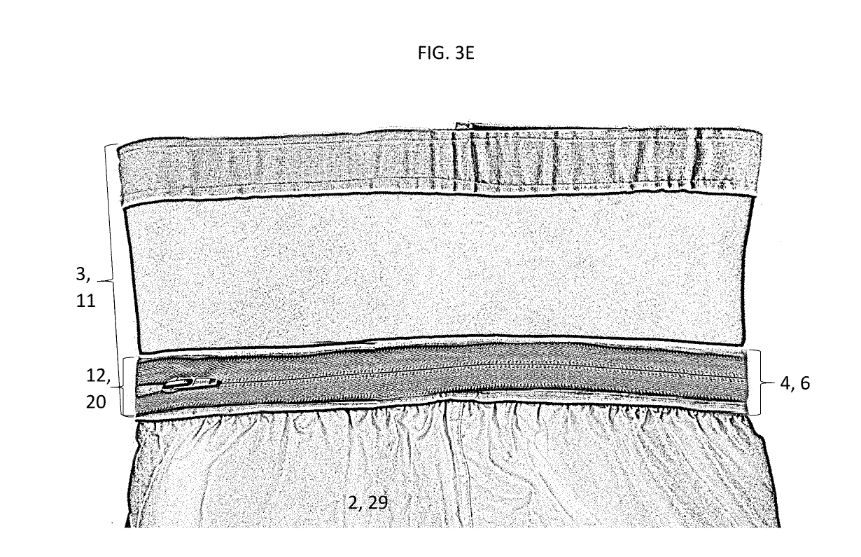

FIG. 3F is a view of the attachment of the wrap to the pants, said attachment being buttons;

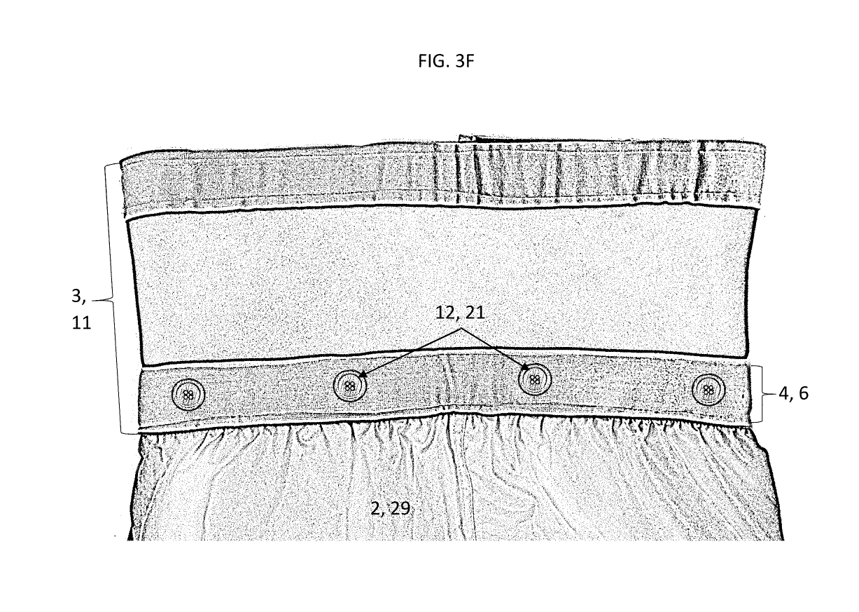

FIG. 4A is a view of the fastener of the wrap, said attachment being a touch fastener;

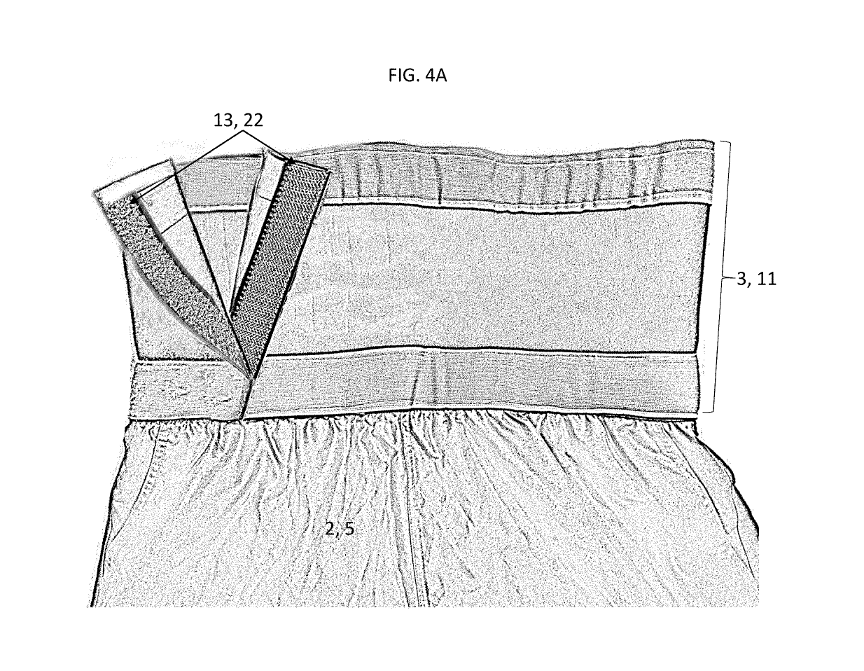

FIG. 4B is a view of the fastener of the wrap, said attachment being a plurality of snaps;

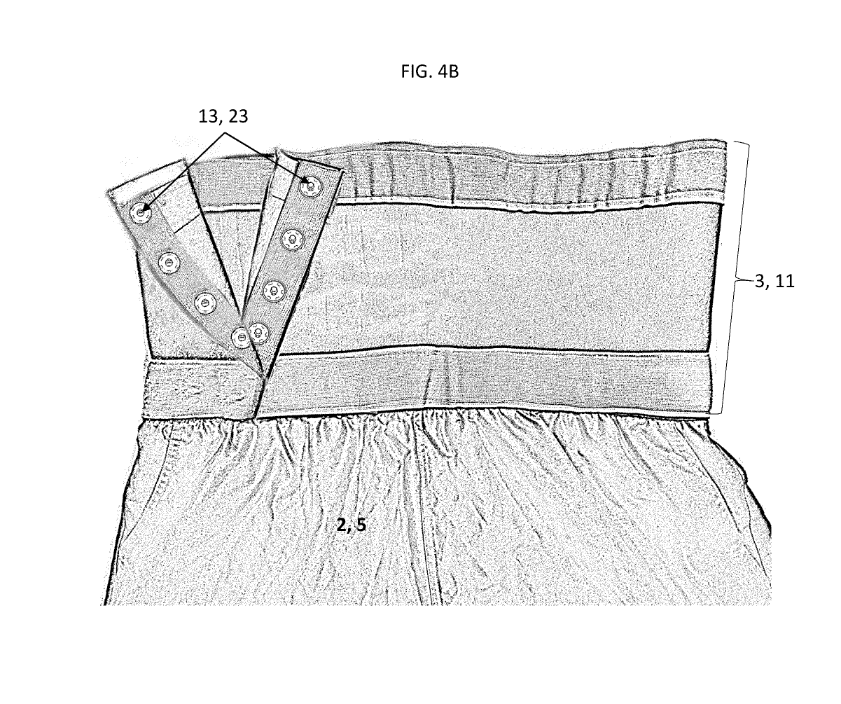

FIG. 4C is a view of the fastener of the wrap, said attachment being a plurality of hooks;

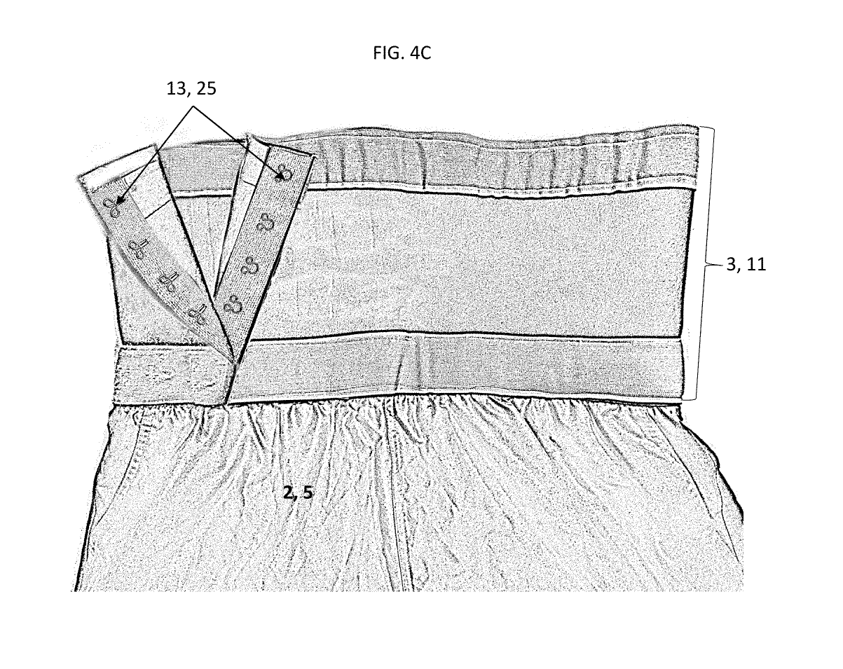

FIG. 4D is a view of the fastener of the wrap, said attachment being a zipper;

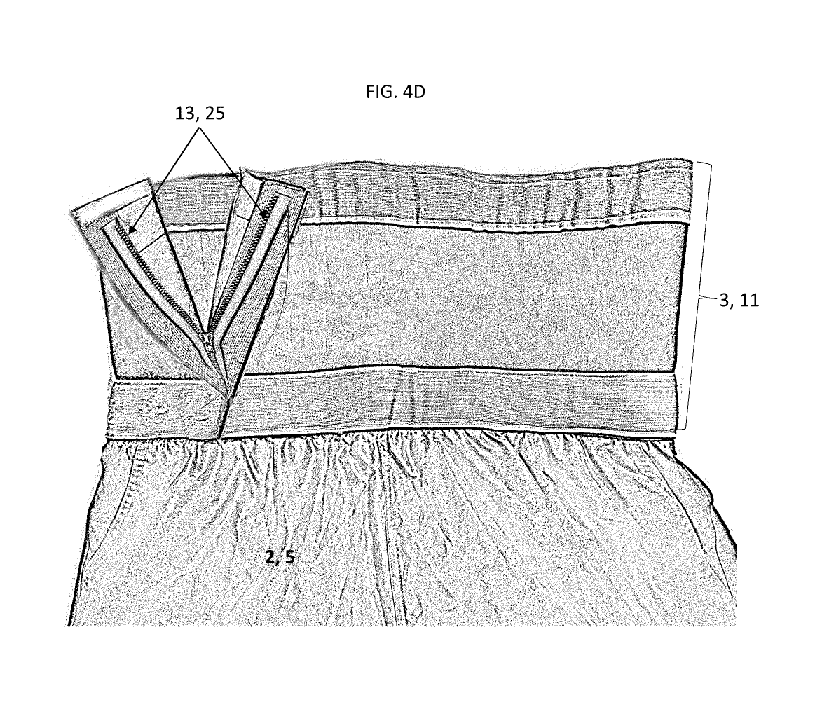

FIG. 4E is a view of the fastener of the wrap, said attachment being a plurality of buttons;

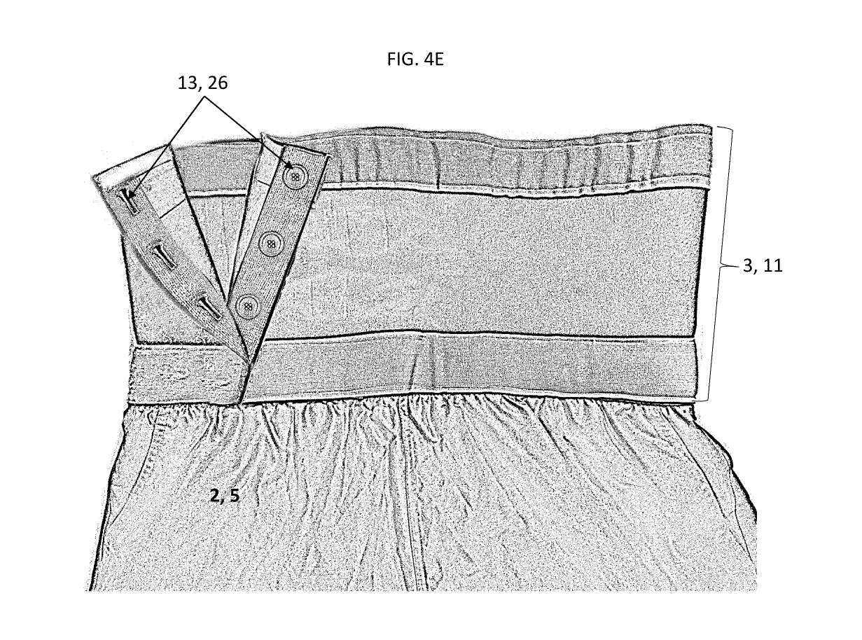

FIG. 5A is a view of in interior of the wrap with the absorbent material in the area of the lower edge and wherein the absorbent material is attached by stitching;

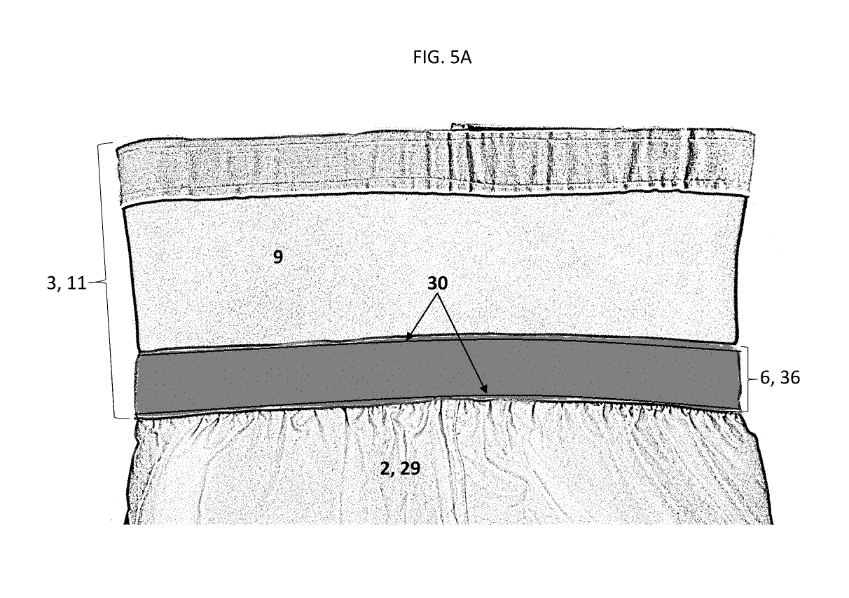

FIG. 5B is a view of in interior of the wrap with the absorbent material in the area of the lower edge and wherein the absorbent material is attached by an adhesive;

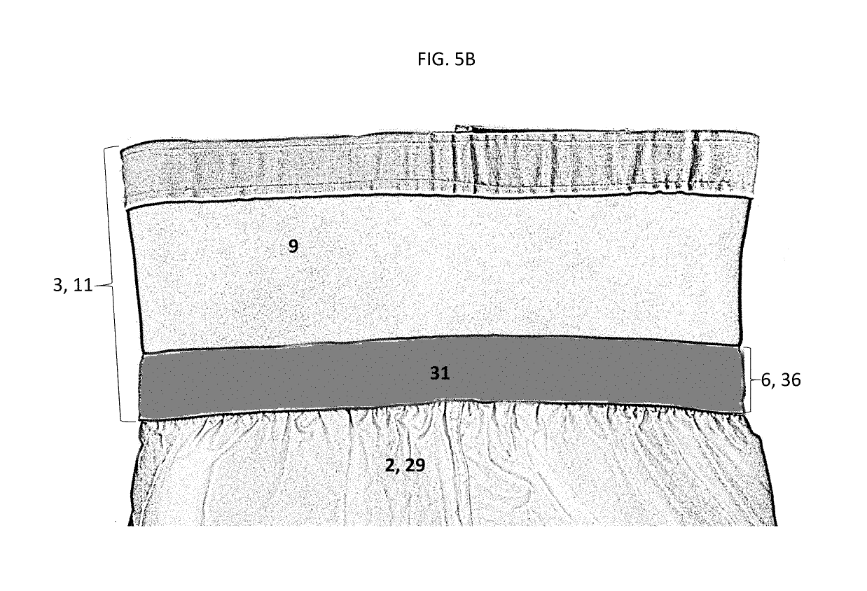

FIG. 5C is a view of in interior of the wrap with the absorbent material in the area of the lower edge and wherein the absorbent material is attached by a touch fastener;

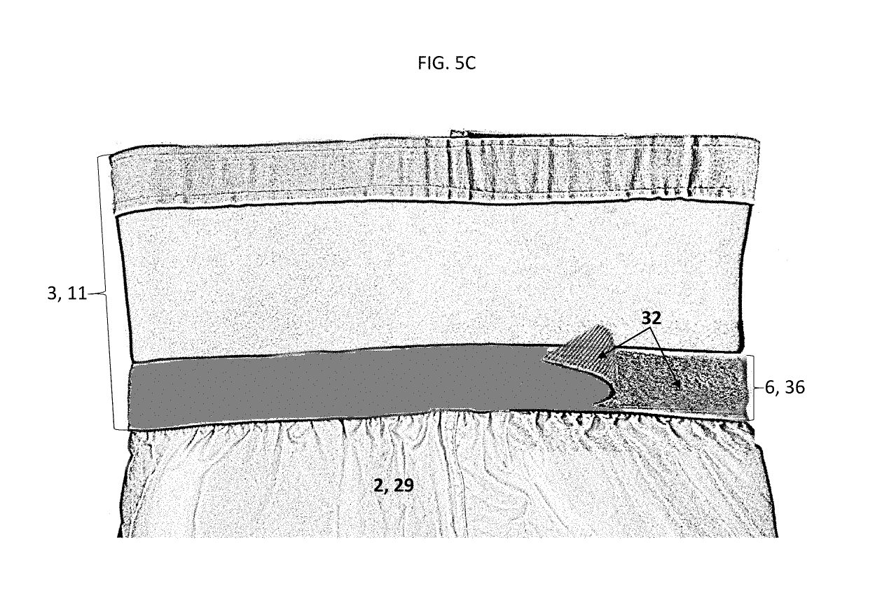

FIG. 5D is a view of in interior of the wrap with the absorbent material in the area of the lower edge and wherein the absorbent material is attached by a plurality of snaps;

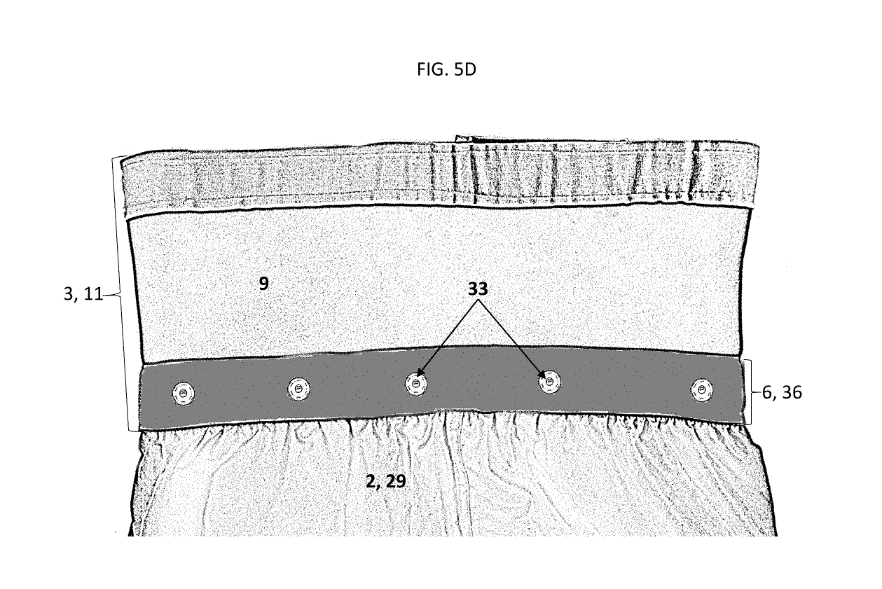

FIG. 5E is a view of in interior of the wrap with the absorbent material in the area of the lower edge and wherein the absorbent material is attached by a zipper; and

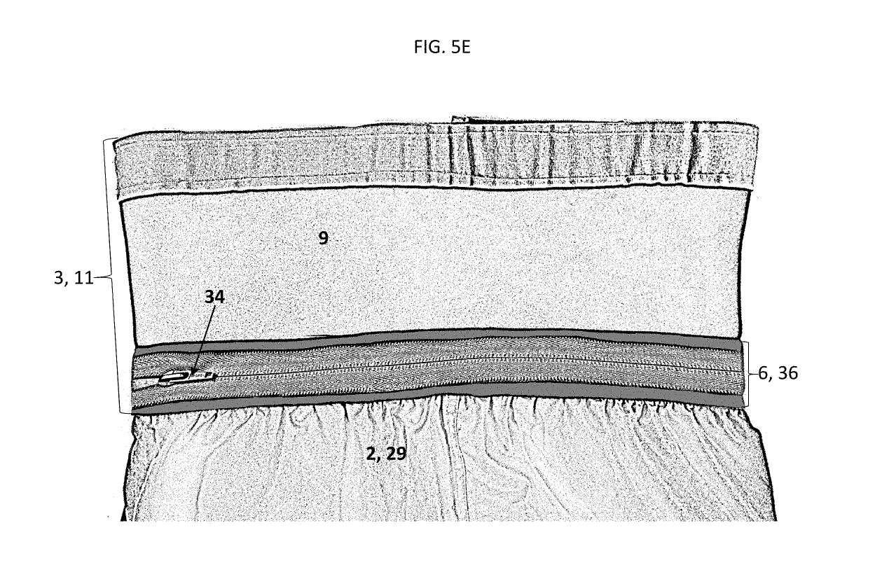

FIG. 5F is a view of in interior of the wrap with the absorbent material in the area of the lower edge and wherein the absorbent material is attached by a plurality of buttons.

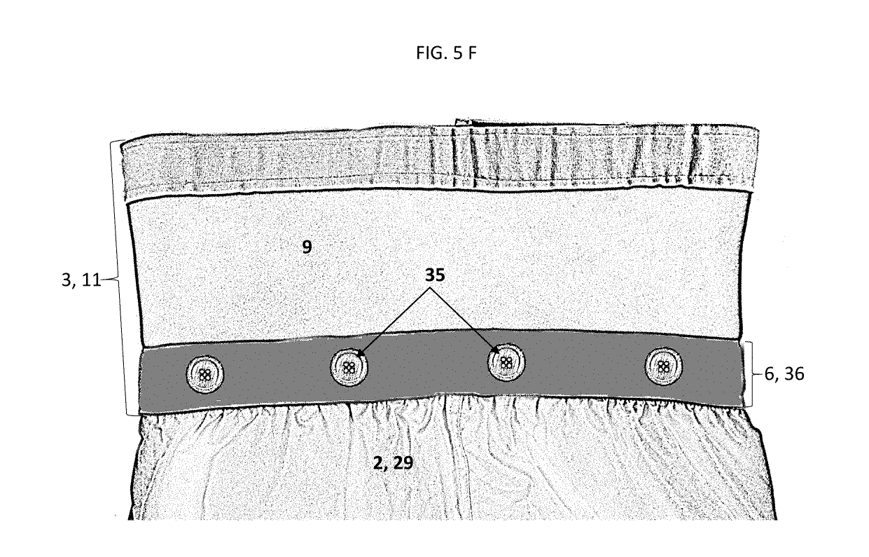

### DETAILED DESCRIPTION OF THE INVENTION

The present disclosure reveals pants with an attached wrap 1 for weight loss and toning one's body comprising pants 2 and a wrap 3. The pants 2 comprise an upper edge 4, an inside 29, and a front 5. The wrap 3 comprises a lower edge 6, a left end 7, a right end 8, an inside 9, an outside 10, and a body 11. The lower edge 6 of the wrap 3 is connected to the upper edge 4 area of the pants 2 by an attachment 12 wherein said attachment 12 is selected from at least one of stitching 16, an adhesive 17, a touch fastener 18, a plurality of snaps 19, a zipper 20, or a plurality of buttons 21. The connection of the wrap 3 to the pants 2 is in such a fashion that when the pants 2 are worn by the wearer, the wrap 3 encompasses and surrounds the midriff area of the wearer and where the left end 7 of the wrap 3 and the right end 8 of the wrap 3 coincide with the front 5 of the pants 2. The wrap 3 is closed around the wearer by stretching the body 11 of the wrap 3 so that it encompasses the midriff of the wearer and at least one of the left end 7 of the wrap 3 overlaps the right end 8 of the wrap 3 or the right end 8 of the wrap 3 overlaps the left end 7 of the wrap 3. The wrap 3 is attached to itself with the use of a fastener 13, said fastener 13 comprising at least one of a touch fastener 22, a plurality of snaps 23, a plurality of hooks 24, a zipper 25, or a plurality of buttons 26.

In the first embodiment 14 of the wrap 3 the wrap 3 is comprised of an impermeable elastic material 27 that is comprised of a at least one of stretchable fabric, a blend of stretchable materials, or layers of stretchable materials, each being common in the industry, including but not limited to sponge neoprene.

In the second embodiment 15 of the wrap 3 the wrap 3 is comprised of a permeable elastic material 28 with pores wherein the permeable material would allow air to pass through the pores and water, sweat, or other material to pass through or be absorbed into the pores. The permeable elastic material 28 is further comprised of a at least one of stretchable fabric, a blend of stretchable materials, or layers of stretchable materials, each being common in the industry such as but not limited to woven polyester.

In either embodiment of the pants and attached wrap 1, on the inside 9 of the wrap 3 in the area of the lower edge 6 there may be an absorbent band 15 for the purpose of absorbing the seat of the wearer, said absorbent band 15 being attached to the inside 9 of the wrap 3 by means of at least one of stitching 30, an adhesive 31, a touch fastener 32, a plurality of snaps 33, a zipper 34, or a plurality of buttons 35.

What is claimed:

1. Pants with an attached wrap for weight loss and toning one's body comprising:
pants and a wrap;
the pants comprising an upper edge and a front;
the wrap comprising an impermeable elastic material a lower edge, a left end, a right end, an inside, an outside, and a body;
the impermeable material is further comprised of a at least one of stretchable fabric, a blend of stretchable materials, or layers of stretchable materials, each being common in the industry;
the lower edge of the wrap being connected to the upper edge area of the pants by an attachment wherein said attachment is selected from at least one of stitching, an adhesive, a touch fastener, a plurality of snaps, a zipper, or a plurality of buttons;
wherein the connection of the wrap to the pants is in such a fashion that when the pants are worn by the wearer, the wrap encompasses and surrounds the midriff area of the wearer and there the left end of the wrap and the right end of the wrap coincide with the front of the pants;
the wrap is closed around the wearer by stretching the body of the wrap so that it encompasses the midriff of the wearer and at least one of the left end of the wrap overlaps the right end of the wrap or the right end of the wrap overlaps the left end of the wrap; and
the wrap is attached to itself with the use of a fastener, said fastener comprising at least one of a touch fastener, a plurality of snaps, a plurality of hooks, a zipper, or a plurality of buttons.

2. The pants and attached wrap of claim 1 wherein, on the inside of the wrap in the area of the lower edge is an absorbent band, said absorbent band being attached to the inside of the wrap by means of at least one of stitching, an adhesive, a touch fastener, a plurality of snaps, a a zipper, or a plurality of buttons.

3. Pants with an attached wrap for weight loss and toning one's body comprising:
pants and a wrap;
the pants comprising an upper edge and a front;
the wrap comprising a permeable elastic material with pores, a lower edge, a left end, a right end, an inside, an outside, and a body;
the permeable material is further comprised of a at least one of stretchable fabric, a blend of stretchable materials, or layers of stretchable materials, each being common in the industry;
the lower edge of the wrap being connected to the upper edge area of the pants by an attachment wherein said attachment is selected from at least one of stitching, an adhesive, a touch fastener, a plurality of snaps, a zipper, or a plurality of buttons;
wherein the connection of the wrap to the pants is in such a fashion that when the pants are worn by the wearer, the wrap encompasses and surrounds the midriff area of the wearer and there the left end of the wrap and the right end of the wrap coincide with the front of the pants;
the wrap is closed around the wearer by stretching the body of the wrap so that it encompasses the midriff of the wearer and at least one of the left end of the wrap overlaps the right end of the wrap or the right end of the wrap overlaps the left end of the wrap; and
the wrap is attached to itself with the use of a fastener, said fastener comprising at least one of a touch fastener, a plurality of snaps, a plurality of hooks, a zipper, or a plurality of buttons.

4. The pants and attached wrap of claim 3 wherein, on the inside of the wrap in the area of the lower edge is an absorbent band, said absorbent band being attached to the inside of the wrap by means of at least one of stitching, an adhesive, a touch fastener, a plurality of snaps, a a zipper, or a plurality of buttons.
